(19) United States
(12) Patent Application Publication
(10) Pub. No.: US 2005/0069532 A1
(43) Pub. Date: Mar. 31, 2005

(54) Treatment of bacterial infection with elastase

(76) Inventors: Yvette Weinrauch, New York, NY (US); Arturo Zychlinsky, Berlin (DE)

Correspondence Address: Michael L Goldman;Nixon Peabody, Clinton Square, Post Office Box 31051, Rochester, NY, 14603-1051, US

(21) Appl. No.: 10/502,895

(22) PCT Filed: Jan. 29, 2003
(86) PCT No.: PCT/US03/02806

Related U.S. Application Data
(60) Provisional application No. 60/353,414, filed on Jan. 31, 2002.

(51) Int. Cl. A61K 38/48

(52) U.S. Cl. 424/94.63

(57) ABSTRACT

The present invention relates to methods of treating bacterial infection in a subject, degrading bacterial virulence factors, preventing bacteria from escaping phagosomes of neutrophils, and preventing bacteria from invading host cells, by use of an elastase.

This application claims benefit of U.S. Provisional Patent Application Ser. No. 60/353,414, filed Jan. 31, 2002.

The subject matter of this application was made with support from the United States National Institutes of Health Grant Nos. AI37720, AI42780, and DK5472. The United States Government may have certain rights.

### FIELD OF THE INVENTION

The present invention relates to methods of treating bacterial infection in a subject, degrading bacterial virulence factors, preventing bacteria from invading host cells, and preventing bacteria from escaping phagosomes of neutrophils, by use of an elastase.

### BACKGROUND OF THE INVENTION

Historically, subjects infected with pathogenic bacteria have been treated with various types of antibiotics. However, drug-resistant infectious agents—those that are not killed or inhibited by antimicrobial compounds—are an increasingly important public health concern. Tuberculosis, gonorrhea, enteric infections, and childhood ear infections are just a few of the diseases that have become more difficult to treat due to the emergence of drug-resistant pathogens. Antimicrobial resistance is becoming a factor in virtually all hospital-acquired (nosocomial) infections. Many physicians are concerned that several bacterial infections soon may be untreatable.

In addition to its adverse effect on public health, antimicrobial resistance contributes to higher health care costs. Treating resistant infections often requires the use of more expensive or more toxic drugs and can result in longer hospital stays for infected patients. The Institute of Medicine, a part of the National Academy of Sciences, has estimated that the annual cost of treating antibiotic resistant infections in the United States may be as high as $30 billion.

A key factor in the development of antimicrobial resistance is the ability of infectious organisms to adapt quickly to new environmental conditions. Microbes generally are unicellular creatures that, compared with multicellular organisms, have a small number of genes. Even a single random gene mutation can have a large impact on their disease-causing properties; and since most microbes replicate very rapidly, they can evolve rapidly. Thus, a mutation that helps a microbe survive in the presence of an antibiotic drug will quickly become predominant throughout the microbial population. Microbes also commonly acquire genes, including those encoding for resistance, by direct transfer from members of their own species or from unrelated microbes.

The innate adaptability of microbes is complemented by the widespread and sometimes inappropriate use of antibiotics. Ideal conditions for the emergence of drug-resistant microbes result when drugs are prescribed for the common cold and other conditions for which they are not indicated or when individuals do not complete their prescribed treatment regimen. Hospitals also provide a fertile environment for drug-resistant pathogens. Close contact among sick patients and extensive use of antimicrobials force pathogens to develop resistance.

Antimicrobial resistance has been recognized since the introduction of penicillin nearly 50 years ago when penicillin-resistant infections caused by Staphylococcus aureus rapidly appeared. Today, hospitals worldwide are facing unprecedented crises from the rapid emergence and dissemination of other microbes resistant to one or more antimicrobial agents.

Diarrheal diseases cause almost 3 million deaths a year—mostly in developing countries, where resistant strains of highly pathogenic bacteria such as Shigella dysenteriae, Campylobacter, Vibrio cholerae, Escherichia coli, and Salmonella are emerging. Recent outbreaks of Salmonella food poisoning have occurred in the United States. A potentially dangerous “superbug” known as Salmonella typhimurium, which is resistant to ampicillin, sulfa, streptomycin, tetracycline, and chloramphenicol, has caused illness in Europe, Canada, and the United States.

The present invention is directed to overcoming these problems in the art.

### SUMMARY OF THE INVENTION

One aspect of the present invention relates to a method treating bacterial infection in a subject. This involves administering an elastase to the subject under conditions effective to target virulence factors from pathogenic bacteria.

Another aspect of the present invention relates to a method of degrading bacterial virulence factors. This involves subjecting the bacterial virulence factors to an elastase under conditions effective to degrade the bacterial virulence factors.

A further aspect of the present invention pertains to a method of preventing bacteria from escaping phagosomes of neutrophils. This is achieved by subjecting the bacteria to an elastase under conditions effective to prevent the bacteria from escaping the phagosomes of the neutrophils.

Another aspect of the present invention is directed to a method of preventing bacteria from invading host cells. This is carried out by subjecting the bacteria to an elastase under conditions effective to prevent the bacteria from invading host cells.

Dissecting how pathogenic bacteria are disarmed by neutrophils, applicants have shown that neutrophil elastase can destroy bacterial virulence factors with high specificity. Neutrophil elastase appears to be the first mammalian protein that is able to distinguish between virulence factors and other bacterial proteins. This discovery suggests the use of neutrophil elastase or derivatives as “smart antibiotics” that would target only bacteria expressing virulence factors.

All available antibiotics target essential functions of bacterial physiology like protein synthesis or cell wall biosynthesis. Hence, these antibiotics attack both pathogens and normal flora. The proposed development of a new generation of antibiotics based on neutrophil elastase recognition of virulence factors would only inactivate disease-causing bacteria. This is significant in view of growing and widespread resistance to antibiotics currently used and the threat of encountering pathogens through intentional release (i.e. bioterrorism).

### BRIEF DESCRIPTION OF THE DRAWINGS

FIGS. 1A-C show a human neutrophil extract enriched in granule proteins (hNEGP) targets virulence proteins of Shigella. FIG. 1A shows that hNEGP kills Shigella at high concentrations. Bacterial viability of 10⁸ CFU of wild-type Shigella flexneri (M90T) after a 30 min incubation at 37° C. decreases at concentrations above 1% V/V of hNEGP. The bar indicates the sublethal concentrations of hNEGP that were used in immunoblot analysis shown in FIG. 1B. FIG. 1B shows that sub-lethal concentrations of hNEGP decrease Shigella virulence proteins. Proteins were precipitated from human neutrophil pellets and filtered culture supernatants, resolved by SDS-PAGE, and analyzed by immunoblotting. The type III secreted proteins Ipa A, B, and C show a substantial decrease (at 0.05% v/v) in the supernatant but not in the intracellular fraction. The secreted (supernatant) and outer membrane (pellet) form of IcsA are also degraded at 0.05% v/v. FIG. 1C shows that bacterial cell integrity is not compromised at sub-lethal concentrations of hNEGP. Immunoblot analysis of cell pellets, with specific antibodies to the OmpA, MBP, and RecA, which are outer membrane, periplasmic and cytosolic marker proteins, respectively, show no hNEGP dependant degradation.

FIGS. 2A-E show that neutrophil elastase degrades Shigella virulence proteins. FIG. 2A shows that treatment of hNEGP with the serine protease inhibitor, PMSF (1 mM), blocked degradation of secreted IpaA, B, C, and the outer membrane (120 kDa) and secreted (90 kDa) forms of IcsA. FIG. 2B shows that Shigella virulence proteins are degraded by neutrophil elastase in hNEGP. Shigella (10⁸ CFU) were incubated with hNEGP, pretreated with the neutrophil elastase specific chemical inhibitors, MeOSuc-AAPV-cmk, ICI-200355, or the physiologic inhibitor, SLPI (1 mM, 20 μM and 750 nM, respectively, for 20 min, at room temperature). Degradation of IpaB and IcsA was blocked by these inhibitors as shown by immunoblot analysis. As shown in FIG. 2C, purified neutrophil elastase, but not cathepsin G, another neutral protease abundant in neutrophil granules, specifically cleaved Shigella virulence proteins. 10⁸ CFU of Shigella were incubated with purified neutrophil elastase or CG at the indicated concentrations. After 30 min incubation, proteins from culture supernatants and cell pellets were analyzed by immunoblot analysis. OmpA was tested in cell pellets as a negative control. FIG. 2D shows that neutrophil elastase does not degrade the type III secretion apparatus. Immunoblots of bacterial pellets treated as in (FIG. 2C) were probed with antibodies to the Shigella (MxiA and MxiD) or Salmonella (InvG) type III proteins. IcsA and OmpA were the positive and negative controls for neutrophil elastase targets in the outer membrane. As shown in FIG. 2E, Shigella virulence factors are degraded by neutrophil elastase in intact neutrophils. Neutrophils 1×10⁶/ml), pretreated with ICI-200355 (20 μM), were infected with wild type Shigella (100 bacteria/neutrophil). At the indicated time points, proteins from the filtered culture supernatants (IpaA and IpaB) or the bacterial pellets (IcsA, RecA, OmpA) were analyzed by immunoblot.

FIGS. 3A-C show that neutrophil elastase degrades secreted virulence proteins of Gram-negative bacterial pathogens. FIG. 3A shows selective degradation of virulence proteins in Shigella supernatants. Secreted proteins from Shigella were incubated with 3.4 nM of purified neutrophil elastase for the indicated times in minutes. Precipitated proteins were separated by SDS-PAGE and stained with Coomassie blue. Proteins identified by MALDI-TOF mass spectrometry are indicated (* indicate discrete cleavage products). As shown in FIG. 3B, neutrophil elastase preferentially degrades virulence proteins in Salmonella. Secreted proteins from wild type Salmonella typhimurium (strain SL1344) were incubated with purified neutrophil elastase and analyzed as described in FIG. 3A. Precipitated proteins were resolved by SDS-PAGE and stained with silver nitrate before MALDI-TOF mass spectrometry analysis. FIG. 3C shows that neutrophil elastase degrades virulence proteins from S. typhimurium and Yersinia enterocolitica (strain W22703). Secreted proteins from wild type S. typhimurium or Y. enterocolitica were incubated with purified neutrophil elastase as described in (FIG. 3A), and examined by immunoblot analysis with specific antibodies for SipC in S. typhimurium and YopB, D, and E in Y. enterocolitica. (−) indicates incubation without neutrophil elastase.

FIG. 4A-I shows that the abrogation of neutrophil elastase permits Shigella to escape the phagosome of neutrophils. Bacteria (labeled B) are contained within vacuoles surrounded by vacuolar membranes (arrows) in FIG. 4A (Human neutrophils infected with wild type Shigella), FIG. 4B (avirulent strain), FIG. 4C (Wild type murine neutrophils infected with wild type Shigella) and FIG. 4E (Murine neutrophil elastase null neutrophils infected with avirulent strain). Bacteria are free in the host cytoplasm in FIG. 4C (Human neutrophils pretreated with the neutrophil elastase inhibitor ICI-200355 (20 μM) before infection with wild type Shigella) and FIG. 4F (Murine neutrophil elastase null neutrophils infected with wild type Shigella). Arrowheads point to the double membrane characteristic of enterobacteria (N) Neutrophil nuclei; Bars=1 μm; cells were fixed 30 min post-infection. FIG. 4G shows that there is increased intracellular survival of Shigella in human neutrophils where neutrophil elastase is blocked. 5×10⁶ neutrophils, preincubated with (+) or without (−) ICI-200355 (20 μM), were infected (10 bacteria/neutrophil) with wild type Shigella in duplicate. After 15 min, the neutrophils from one sample were washed and cell associated CFU were determined. The second sample was incubated with gentamicin (100 μg/ml) for an additional 30 min to kill the extracellular bacteria. The neutrophils were then washed and intracellular CFU were determined. The values reflect the ratio of intracellular bacteria (samples incubated with gentamicin) to intracellular+membrane-attached bacteria (samples incubated without gentamicin). FIG. 4H shows increased intracellular survival of Shigella in murine neutrophil elastase null neutrophils. Peritoneal neutrophils from null mice and isogenic controls were infected with wild type Shigella as described in FIG. 4G. FIG. 4I shows increased cytotoxicity of Shigella in human neutrophils when neutrophil elastase is blocked. At all moi tested, pretreatment of neutrophils with the neutrophil elastase inhibitor ICI-200355 (20 μM) before infection with wild type Shigella (⋄) resulted in more cell death than wild type Shigella infection in control neutrophils (□). Noninvasive Shigella control strains caused no cell death regardless of whether neutrophils were pretreated with ICI-200355 before infection (Δ) or not (◯). Cytotoxicity was determined by release of cytoplasmic lactate dehydrogenase (LDH) after 2 h incubation. Data in FIGS. 4G-I are the mean and SD of triplicates and are representative of a minimum of three experiments with similar results.

### DETAILED DESCRIPTION OF THE INVENTION

One aspect of the present invention relates to a method treating bacterial infection in a subject. This involves administering an elastase to the subject under conditions effective to target virulence factors from pathogenic bacteria.

Neutrophils play a central role in host defenses against invading microorganisms. Within hours, activated neutrophils migrate to the site of infection where they release their granule derived antimicrobial products. Amongst these products is neutrophil elastase (also known leukocyte elastase) which is a 30 kDa glycoprotein chymotrypsin-like, serine proteinase. It can be found in the azurophilic granules of the neutrophils (Enzyme No. E.C.3.4.32.37 of the Enzyme Commission of the International Union of Biochemistry) and in sputum leukocytes (Enzyme No. E.C.3.4.21.37 of the Enzyme Commission of the International Union of Biochemistry, available commercially from Elastin Products Co., Inc., Owensville, Mo.).

The elastase used in accordance with the present invention can be whole or full length elastase or an active component part of elastase. In general, elastases have a triad of conserved amino acid residues within its catalytic domain, which degrades insoluble elastin into soluble peptides by cleaving carboxy terminal bonds (particularly bonds having valine at the P1 position) to small, hydrophobic residues. Owen, et. al., “The Cell Biology of Leukocyte-Mediated Proteolysis,” J. Leuk. Biol. 65: 137-50 (1999)), which is hereby incorporated by reference in its entirety. The carbohydrate side chains are joined together by 4 disulfide bonds. Sinha, et al., “Primary Structure of Human Neutrophil Elastase,” Proc. Nat'l Acad. Sci. USA 84: 2228-32 (1987), which is hereby incorporated by reference in its entirety. The triad of conserved amino acid residues includes His-41, Asp-88, and Ser-173. The serine at the active site is highly nucleophilic and has a high affinity for small uncharged amino acids. Lee, et al., “State of the Art: Leukocyte Elastase—Physiological Functions and Role in Acute Lung Injury,” Am J. Respir. Crit. Care Med. 164: 896-904 (2001), which is hereby incorporated by reference in its entirety. These residues are widely separated in the primary sequence but are brought together at the active site of the enzymes in their tertiary structure. Owen, et. al., “The Cell Biology of Leukocyte-Mediated Proteolysis,” J. Leuk. Biol. 65: 137-50 (1999), which is hereby incorporated by reference in its entirety. The human leukocyte elastase is a single chain polypeptide with 218 amino acid residues and contains 2 asparagine-linked carbohydrate side chains. In addition to full length elastase, the elastase can be used in accordance with the present invention in the form of just the His-Asp-Ser catalytic domain or carbohydrate side of elastase.

The pathogenic bacteria treated in accordance with this aspect of the present invention can be enterobacteria, such as a Shigella species, a Salmonella species, or a Yersinia species. Bacterial infection by other species, such as a Chlamydia species, Pseudomonas aeruginosa, or a plant pathogenic bacteria, can also be treated in accordance with the present invention.

Elastase may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active materials may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These active compounds may also be administered parenterally. Solutions or suspensions of these active materials can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The elastase thereof may also be administered directly to the airways in the form of an aerosol. For use as aerosols, the material of the present invention in solution or suspension may be packaged in a pressurized aerosol container together with suitable propellants, for example, hydrocarbon propellants like propane, butane, or isobutane with conventional adjuvants. The elastase also may be administered in a non-pressurized form such as in a nebulizer or atomizer.

Preferably, a concentration of at least 1.2 nM of the elastase is used to treat bacterial infection pursuant to the present invention. At this concentration, elastase is useful in treating bacterial infection either in vivo or in vitro.

Another aspect of the present invention relates to a method of degrading bacterial virulence factors. This involves subjecting the bacterial virulence factors to an elastase under conditions effective to degrade the bacterial virulence factors. This aspect of the present invention is carried out by administering the elastase in substantially the same form, manner, and concentration to treat virulence factors from the same bacteria as described above. In accordance with this aspect of the present invention, the act of subjecting targets virulence factors from pathogenic bacteria inactivates the bacteria. This method can be carried out in vivo or in vitro.

A further aspect of the present invention pertains to a method of preventing bacteria from escaping phagosomes of neutrophils. This is achieved by subjecting the bacteria to an elastase under conditions effective to prevent the bacteria from escaping the phagosomes of the neutrophils. Again, this aspect of the present invention is carried out by administering the elastase in substantially the same form, manner, and concentration to prevent the same bacteria escaping phagosomes of neutrophils, as described above. This method can be carried out in vivo or in vitro. By subjecting bacteria to elastase in accordance with this aspect of the present invention, virulence factors from pathogenic bacteria are targeted and the bacteria are inactivated.

Another aspect of the present invention is directed to a method of preventing bacteria from invading host cells. This is carried out by subjecting the bacteria to an elastase under conditions effective to prevent the bacteria from invading host cells. This aspect of the present invention is carried out by administering the elastase in substantially the same form, manner, and concentration to prevent bacterial invasion of host cells, as described above. This method can be carried out in vitro or in vivo. By subjecting bacteria to elastase in accordance with this aspect of the present invention, virulence factors from pathogenic bacteria are targeted and the bacteria are inactivated.

### EXAMPLES

### Example 1

### Bacterial Strains and Growth Conditions

M90T, an invasive isolate of S. flexneri serotype 5, BS176, the noninvasive derivative of M90T and the Shigella ipaD mutant (Menard et al., “Nonpolar Mutagenesis of the ipa Genes Defines IpaB, IpaC, and IpaD as Effectors of Shigella Flexneri Entry into Epithelial Cells,” J Bacteriol 175:5899-5906 (1993), which is hereby incorporated by reference), which constitutively secretes the Ipa proteins was grown to the exponential phase of growth in tryptic soy broth (TSB) with aeration. S. typhimurium, strain SL1344, was grown overnight in LB medium at 37° C. without agitation and Y. enterocolitica (strain W22703), was grown at room temperature to an optical density at OD 600 nm of 0.4 in TSB supplemented with 5 mM EGTA and 20 mM MgCl2. The bacteria were centrifuged and the cell pellet resuspended in nutrient broth supplemented with phosphate-buffered (20 mm, pH 7.4) physiological saline (10⁸ CFU/ml) and cultures shifted to 37° C. for 2 h.

### Example 2

### Bactericidal Activity

Bactericidal activity of hNEGP prepared as described in (Weiss et al., “Purification and Characterization of a Potent Bactericidal and Membrane Active Protein from the Granules of Human Polymorphonucelar Leukoytes,” J. Biol. Chem 253:2664-2672 (1978), which is hereby incorporated by reference) was quantified with wild-type strain M90T. Briefly 108 bacteria were incubated in a total volume of 1 ml for 30 min at 37° C. with shaking as described (Mandic-Mulec et al., “Shigella Flexneri is Trapped in Polymorphonuclear Leukocyte Vacuoles and Efficiently Killed,” Infect Immun 65:110-115 (1997), which is hereby incorporated by reference). After aliquots were removed for determination of colony forming units (CFU), the sample was centrifuged (5 min at 14,000 g). Supernatants were filtered through a 0.2 μm pore-size filter and recovered proteins were precipitated with methanol/chloroform (Lee et al., “Type III Machines of Pathogenic Yersiniae Secrete Virulence Factors into the Extracellular Milieu,” Molec. Micro. 31:1619-1629 (1999), which is hereby incorporated by reference).

### Example 3

### Protein Preparation and Immunoblot Analysis

Protein from bacterial pellets (2.5×10⁷ cell equivalent) and culture supernatants (1 ml) were subjected to SDS-polyacrylamide gel (12.5%) electrophoresis (SDS-PAGE). The protein bands separated by SDS-PAGE were transferred to a nitrocellulose membrane and detected using antibodies specific for IpaA, IpaB, IpaC, and IcsA. Alternatively, secreted proteins (30 μg) from culture supernatants from the indicated strains were filtered after separation by centrifugation of bacterial cultures and treated with 3.4 nM neutrophil elastase after which the reactions were stopped with the addition of PMSF (1 mM) at the indicated times. Proteins were then precipitated as described above, resolved by SDS-PAGE, and stained with Coomassie blue or silver nitrate or subjected to immunoblot analysis with SipC, or YopB, YopD, and YopE antisera. Immunoblotting for type III components were detected in bacterial pellets with anti-MxiD, InvG, and LcrD antibodies. MxiA was detected with the anti-LcrD antibody, since these proteins are homologous (Ginocchio et al., “Functional Conservation Among Members of the Salmonella Typhimurium InvA Family of Proteins,” Infect Immun 63:729-732 (1995), which is hereby incorporated by reference). The LcrD antibody recognizes a band of the right molecular weight in wild type Shigella, which is absent in the avirulent strain BS176, which lacks the plasmid where MxiA is encoded.

### Example 4

### Enzymatic Assays

Neutrophil elastase was quantified in hNEGP using N-methoxysuccinyl-Ala-Ala-Pro-Val-p-nitroanilide as a substrate, according to manufacturer's protocols (Elastin Products Company) and compared to hNEGP pretreated with PMSF (1 mM, rm temp 20 min) or neutrophil elastase inhibitors, MeOSuc-AAPV-cmk (Sigma), ICI-200355 (Huang et al., “Effect of Trifluoromethyl Ketone-Based Elastase Inhibitors on Neutrophil Function in Vitro,” J Leukoc Biol 64:322-330 (1998), which is hereby incorporated by reference) (AstraZeneca Pharmaceuticals), and SLPI (R&D Systems) by comparison with purified, active-site titrated neutrophil elastase. Neutrophil elastase activity in hNEGP was completely inhibited with PMSF, MeOSuc-AAPV-cmk, ICI-200355, or SLPI. CG (Elastin Products Company) was assayed according to manufacturer's instructions. Neutrophil elastase activity of intact neutrophils was determined as previously described (Huang et al., “Effect of Trifluoromethyl Ketone-Based Elastase Inhibitors on Neutrophil Function in Vitro,” J Leukoc Biol 64:322-330 (1998), which is hereby incorporated by reference). Neutrophil elastase did not affect bacterial viability even at 2,000 nM.

### Example 5

### Isolation of Neutrophils

Human neutrophils (>95% pure) were isolated from peripheral blood of healthy donors using the dextran-Ficoll method (Weiss et al., “Oxygen-Independent Intracellular and Oxygen-Dependent Extracellular Killin of Escherichia Coli S15 by Human Polymorphonuclear Leukocytes,” J. Clin. Invest. 76:206-212 (1985), which is hereby incorporated by reference), and resuspended in complete culture medium (Dulbecco's modified Eagle's medium, 10% fetal bovine serum, CM), prior to stimulation. Murine thyoglycollate elicited peritoneal neutrophils (>80% pure) were isolated from neutrophil elastase −/− or isogenic controls, resuspended at 5×10⁶/ml and allowed to adhere to plastic plates in CM. Human neutrophils were resuspended at 5×10⁶/ml, and allowed to adhere to plastic plates in CM containing the biologically relevant (Sansonetti et al., “Interleukin-8 Controls Bacterial Transepithelial Translocation at the Cost of Epithelial Destruction in Experimental Shigellosis,” Infect Immun 67:1471-1480 (1999), which is hereby incorporated by reference) activator IL-8 (10⁻⁸ M, R&D Systems). After 30 min incubation at 37° C., the medium was replaced with serum free culture medium (SFM), with or without the neutrophil elastase inhibitor ICI-200355 (Huang et al., “Effect of Trifluoromethyl Ketone-Based Elastase Inhibitors on Neutrophil Function in Vitro,” J Leukoc Biol 64:322-330 (1998), which is hereby incorporated by reference) (20 μM), for 30 min before infection. ICI-200355 is not cytotoxic (as measured by lactate dehydrogenase (LDH) release (Promega)) and does not affect ingestion or killing of non-virulent bacteria (Huang et al., “Effect of Trifluoromethyl Ketone-Based Elastase Inhibitors on Neutrophil Function in Vitro,” J Leukoc Biol 64:322-330 (1998), which is hereby incorporated by reference). ICI-200355 blocks 80% of neutrophil elastase activity (Huang et al., “Effect of Trifluoromethyl Ketone-Based Elastase Inhibitors on Neutrophil Function in Vitro,” J Leukoc Biol 64:322-330 (1998), which is hereby incorporated by reference). Human or murine neutrophils were infected with M90T or BS176 in SFM centrifuged at 700 g for 10 min and then incubated at 37° C. and 5% CO₂. Thirty min post-infection neutrophils were washed, fixed and processed for TEM using standard methods. For bacterial viability, duplicate samples were plated. One sample was taken 15 min after incubation to determine the number of cell-associated bacteria and the second sample after a 30 min incubation with gentamicin (100 μg/ml), to determine the number of intracellular bacteria. Infections were performed in SFM, because Shigella invasion is independent of serum opsonins (Gbarah et al., “Shigella Flexneri Transformants Expressing Type 1 (Mannose-Specific) Fimbriae Bind To, Activate, and are Killed by Phagocytic Cells,” Infect. Immun. 61:1687-1693 (1993), which is hereby incorporated by reference). A million neutrophils were infected with wild type Shigella (10⁸) in a total volume of 1 ml to test the degradation of Shigella virulence factors in infections. At the indicated times, the proteins in filtered culture supernatants were precipitated as described above. Cell pellets (neutrophils and bacterial) or supernatant proteins were solubilised in SDS sample buffer and processed for immunoblot analysis.

Neutrophils play a central role in host defenses against invading microorganisms. In the phagolysosome of neutrophils, bacteria are exposed to enzymes, antibacterial polypeptides, oxygen radicals, and low pH (Klebanoff, S. J., “Inflammation: Basic Principles and Clinical Correlates” (eds. Gallin, J. I. & Snyderman, R.) 721-768 (Lippincotte Williams & Wilkens, Philadelphia, 1999); Elsbach et al., “Inflammation: Basic Principles and Clinical Correlates,” (eds. Gallin, J. I. & Snyderman, R.) 801-817 (Lippincotte Williams & Wilkens, Philadelphia, 1999), which are hereby incorporated by reference). In contrast to other host cell types, neutrophils prevent Shigella from escaping the phagosome although the bacteria are viable for up to one hour in this compartment (Mandic-Mulec et al., “Shigella Flexneri is Trapped in Polymorphnuclear Leukocyte Vacuoles and Efficiently Killed,” Infect Immun 65:110-115 (1997), which is hereby incorporated by reference). These findings prompted applicants' hypothesis that neutrophils destroyed the virulence factors of Shigella before bacterial viability was affected. To mimic in vivo conditions in vitro, different concentrations of a human neutrophil extract enriched in granule proteins (hNEGP) was tested on Shigella.

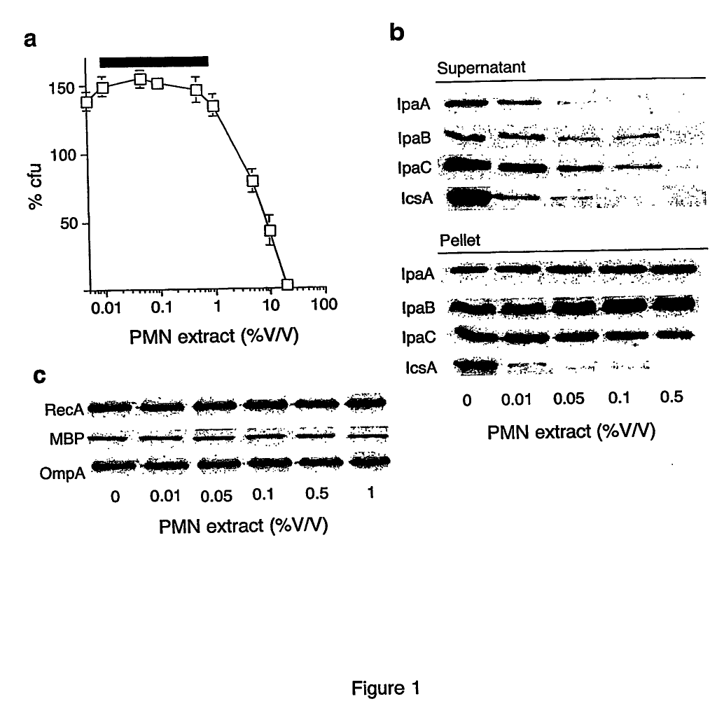

Shigella was effectively killed at high concentrations of human hNEGP, but concentrations below 1% did not affect bacterial viability (FIG. 1A). Sublethal concentrations of hNEGP (up to 0.5% vol/vol) substantially decreased the levels of type III secreted proteins, IpaA, B, and C, in culture supernatants (FIG. 1B). This was surprising, because the addition of various other proteins increase type III secretion (Bahrani et al., “Secretion of Ipa Proteins by Shigella Flexneri: Inducer Molecules and Kinetics of Activation,” Infect. Immun 65:4005-4010 (1997), which is hereby incorporated by reference). The intracellular pool of these proteins was not affected (FIG. 1B). In addition, levels of the 120-kDa outer membrane (bacterial pellets) and the 90-kDa secreted (culture supernatants) forms of IcsA were also decreased at hNEGP concentrations as low as 0.01% vol/vol. Sub-lethal concentrations of hNEGP did not affect outer membrane protein A (OmpA), maltose binding protein (MBP), or recombinase A (RecA), which are outer membrane, periplasmic and cytosolic proteins respectively, confirming that bacterial cell integrity was maintained (FIG. 1C). Interestingly, OmpA and IcsA occupy the same subcellular compartment, suggesting that hNEGP targeted virulence proteins.

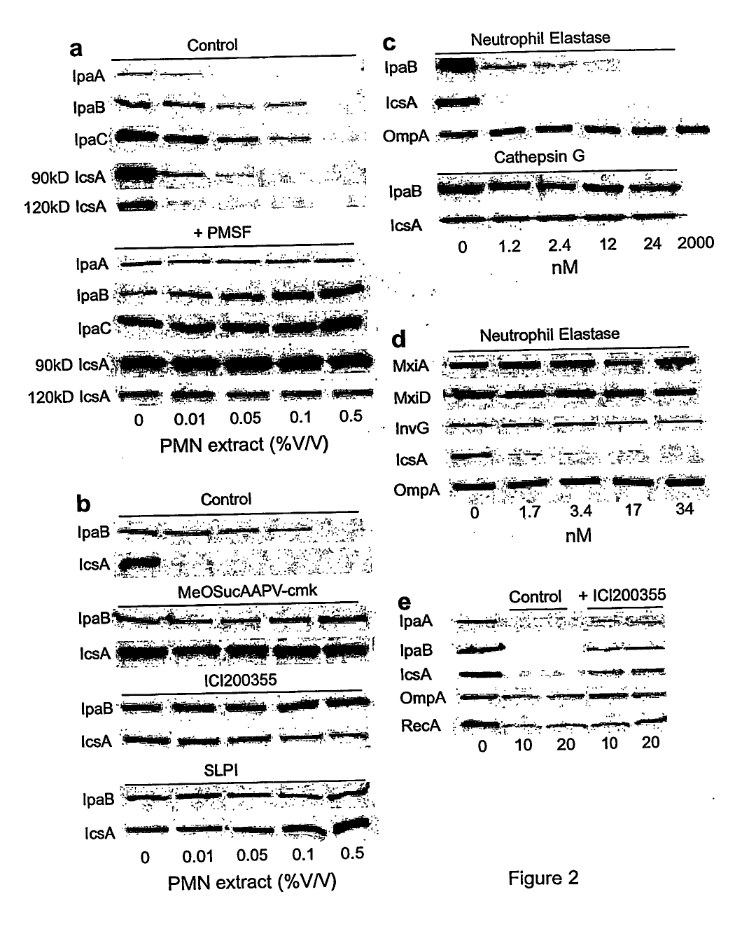

To test whether neutral serine proteases, which are abundant in neutrophil granules (Owen et al., “The Cell Biology of Leukocyte-Mediated Proteolysis,” J Leukoc Biol 65:137-150. (1999), which is hereby incorporated by reference), were responsible for the degradation of Shigella virulence proteins, applicants used phenylmethylsulfonyl fluoride (PMSF), a group-specific serine protease inhibitor. PMSF blocked the degradation of IpaA, B, and C, as well as the membrane bound and cleaved forms of IcsA (FIG. 2A).

Using specific inhibitors, applicants identified neutrophil elastase as the protease that degrades Shigella effectors. Pretreatment of hNEGP with chemical neutrophil elastase inhibitors (MeOSucAlaAlaProVal-chloromethyl ketone (MeOSucAAPV-cmk) (Campbell et al, “Elastase and Cathepsin G of Human Monocytes. Quantification of Cellular Content, Release in Response to Stimuli, and Heterogeneity in Elastase-Mediated Proteolytic Activity,” J Immunol 143:2961-2968 (1989), which is hereby incorporated by reference), the trifluoromethyl ketone-based ICI-200355 (Veale et al., “Orally Active Trifluoromethyl Ketone Inhibitors of Human Leukocyte Elastase,” J Med Chem 40:3173-3181 (1997), which is hereby incorporated by reference), and the physiologic inhibitor, human Secretory Leukocyte Protease Inhibitor (SLPI) (Wright et al, “Inhibition of Murine Neutrophil Serine Proteinases by Human and Murine Secretory Leukocyte Protease Inhibitor,” Biochem Biophys Res Commun 254:614-617 (1999), which is hereby incorporated by reference), effectively blocked degradation of the Shigella proteins (FIG. 2B, only IpaB and the membrane bound form of IcsA are shown, but IpaA, IpaC and the secreted form of IcsA behaved equivalently). SLPI blocks neutrophil elastase and Cathepsin G but not Proteinase 3, another neutrophil neutral protease (Wright et al, “Inhibition of Murine Neutrophil Serine Proteinases by Human and Murine Secretory Leukocyte Protease Inhibitor,” Biochem Biophys Res Commun 254:614-617 (1999), which is hereby incorporated by reference).

Purified human neutrophil elastase, at concentrations as low as 1.2 nM cleaved Shigella virulence proteins significantly (FIG. 2C, only IpaB and IcsA are shown). This concentration mirrors the amount of neutrophil elastase present in sublethal concentrations of hNEGP. Belaaouaj et al. (Belaaouaj et al., “Degradation of Outer Membrane Protein A in Escherichia Coli Killing by Neutrophil Elastase,” Science 289:1185-1188 (2000), which is hereby incorporated by reference) observed that high concentrations of neutrophil elastase partially digest OmpA of nonpathogenic Escherichia coli (2 μM, 4 h with 106 bacteria). Applicants observed that IcsA was degraded at 1.2 nM of neutrophil elastase (30 min incubation with 108 bacteria). In these conditions, OmpA was not degraded in either Shigella or E. coli, indicating that neutrophil elastase preferentially cleaved proteins expressed exclusively by pathogens FIG. 2C). Neutrophil elastase also cleaves a broad spectrum of matrix macromolecules, proteolytically activates some antimicrobial peptides (Panyutich et al., “Porcine Polymorphonuclear Leukocytes Generate Extracellular Microbicidal Activity by Elastase-Mediated Activation of Secreted Proprotegrins,” Infect Immun 65:978-985 (1997), which is hereby incorporated by reference) and is implicated in various pathological conditions involving tissue injury (Owen et al., “The Cell Biology of Leukocyte-Mediated Proteolysis,” J Leukoc Biol 65:137-150. (1999), which is hereby incorporated by reference). In contrast to neutrophil elastase, purified Cathepsin G, another prominent neutral serine protease in hNEGP, did not degrade the Shigella effectors (FIG. 2C).

Neutrophil elastase did not target the type III secretion apparatus itself, which consists of a basal component and a needle that extends beyond the outer membrane (Tamano et al., “Supramolecular Structure of the Shigella Type III Secretion Machinery: The Needle Part is Changeable in Length and Essential for Delivery of Effectors,” EMBO J. 19:3876-3887 (2000); Blocker et al., “Structure and Composition of the Shigella Flexneri ‘Needle Complex’, a Part of its Type III Secretion,” Mol Microbiol 39:652-663 (2001), which are hereby incorporated by reference). MxiA and MxiD, inner and outer membrane needle proteins, respectively of Shigella, as well as InvG, a type III component of Salmonella, were not degraded by neutrophil elastase (FIG. 2D).

Neutrophil elastase is responsible for the degradation of Shigella virulence factors in intact neutrophils thus confirming the data using hNEGP. IpaA, IpaB and IcsA, but not the cytoplasmic (RecA) or outer membrane (OmpA) markers, were degraded within 10 min of human neutrophil infection with wild type Shigella. This degradation was dependent on neutrophil elastase since the cell permeable NE inhibitor ICI-200355 blocked it (FIG. 2E).

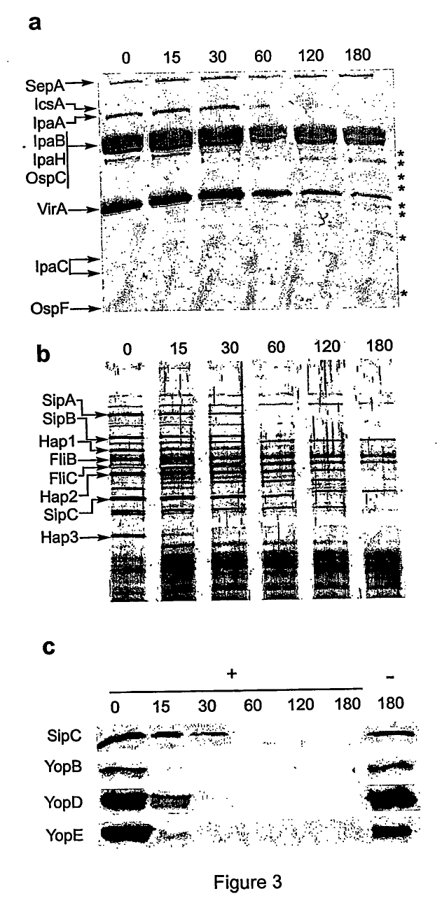

It is not known how neutrophil elastase recognizes its substrates since the only requirement for cleavage is either an alanine or a valine in the P1 position. Purified neutrophil elastase showed specificity by selectively degrading virulence factors in Shigella culture supernatants (FIG. 3A). Identification of the secreted proteins by MALDI-TOF mass spectrometry showed that virulence factors (e.g. VirA and the Ipas) were cleaved, but proteins that have not been associated with virulence (SepA and OspF) remained intact (FIG. 3A). Neutrophil elastase degradation was not dependent upon the amount of substrate since SepA and OspF were present in higher amounts than, for example, the virulence factors IcsA or IpaC. The repertoire of non-virulent proteins is small because wild type Shigella secreted proteins consist mostly of virulence factors, while avirulent strains secrete few proteins. The appearance of discrete cleavage products (* in FIG. 3A) suggested that the Shigella proteins were folded in the culture supernatants and that only certain of the many potential cleavage sites are initially attacked by neutrophil elastase.

Neutrophil elastase also targeted proteins secreted by two other Gram-negative pathogens, Salmonella and Yersinia. The Salmonella virulence proteins, SipA, B, C, and HAPs, required for flagellar structure, (FIGS. 3B, C) as well as the Yersinia virulence proteins, YopB, D and E, were also degraded by neutrophil elastase (FIG. 3C). Interestingly, FliB and FliC in Salmonella, which have recently been shown to be activators of the innate immune receptor TLR5 (Hayashi et al., “The Innate Immune Response to Bacterial Flagellin is Mediated by Toll-Like Receptor 5,” Nature 410:1099-1103 (2001), which is hereby incorporated by reference), were not degraded by neutrophil elastase (FIG. 3B). These results indicate that neutrophil elastase targets virulence proteins from different pathogens. The role of neutrophil elastase in Salmonella and Yersinia infections, as well as the susceptibility of effectors of other pathogenic bacteria, remains to be determined.

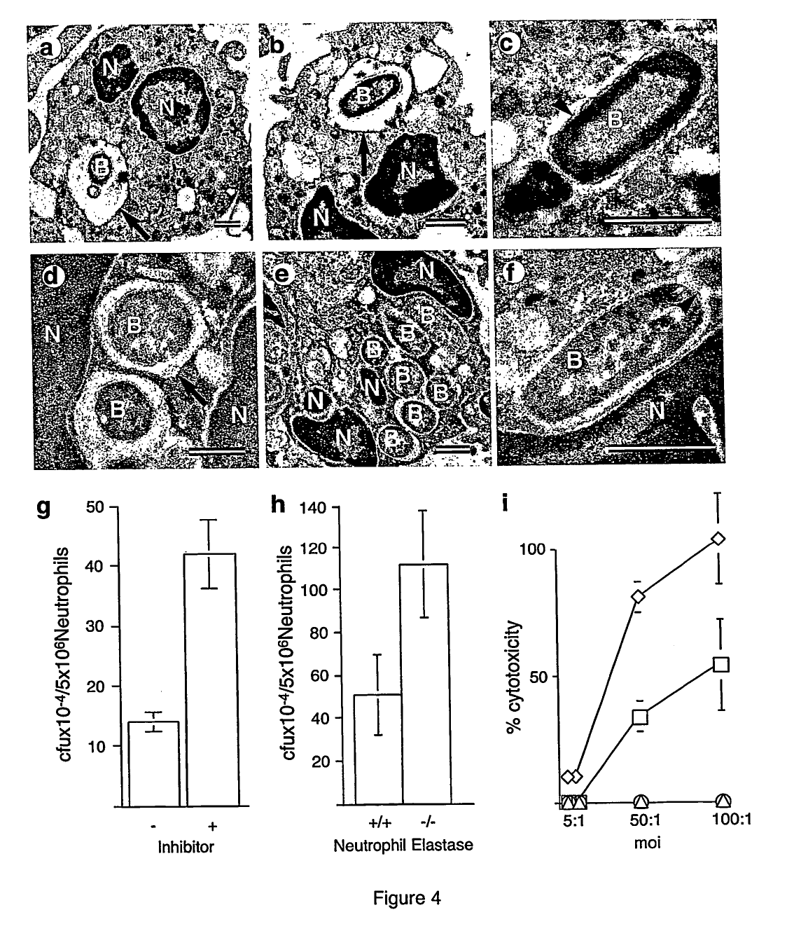

At low multiplicities of infection, Shigella is trapped in the phagolysosome of human neutrophils (FIG. 4A). Based on these results, it is hypothesized that if neutrophil elastase was inactivated, Shigella Ipa proteins would remain functional and the bacteria would escape from the phagosome into the neutrophil cytoplasm. Indeed, when human neutrophils were pre-incubated with ICI-200355 before infection with wild type Shigella, the bacteria were found free in the cytoplasm (FIG. 4C).

Although Shigella is a human specific pathogen and murine neutrophils lack (Eisenhauer et al., “Mouse Neutrophils Lack Defensins,” Infect Immun 60:3446-3447 (1992), which is hereby incorporated by reference) the components of the non-oxidative arsenal of human neutrophils essential to kill Shigella (Mandic-Mulec et al., “Shigella Flexneri is Trapped in Polymorphnuclear Leukocyte Vacuoles and Efficiently Killed,” Infect Immun 65:110-115 (1997), which is hereby incorporated by reference) and other Gram negative bacteria (Elsbach et al., “Inflammation: Basic Principles and Clinical Correlates,” (eds. Gallin, J. I. & Snyderman, R.) 801-817 (Lippincotte Williams & Wilkens, Philadelphia, 1999), which is hereby incorporated by reference), neutrophil elastase null mice were used to test (Belaaouaj et al., “Degradation of Outer Membrane Protein A in Escherichia Coli Killing by Neutrophil Elastase,” Science 289:1185-1188 (2000), which is hereby incorporated by reference) whether in the absence of neutrophil elastase neutrophils could contain Shigella in the phagosome. Whereas Shigella was contained within the phagosome of murine wild type neutrophils (FIG. 4D), in neutrophils from neutrophil elastase −/− mice Shigella were found free in the cytoplasm (FIG. 4F). The visibility of the bacterial double membrane in FIGS. 4C and 4F, but not of the surrounding vacuolar membrane, that the Shigellae were free in the cytoplasm. A non-invasive strain of Shigella used as a control remained in the phagosome in human neutrophils incubated with ICI-200355 (FIG. 4B) or in murine neutrophil elastase −/− neutrophils (FIG. 4E).

Since neutrophils in which neutrophil elastase was inactivated allowed the escape of wild type Shigella into the cytoplasm, a corresponding increase in Shigella survival is anticipated. Shigella survived indeed better both in neutrophils treated with ICI-200355 and in neutrophil elastase null neutrophils than in controls (FIGS. 4G and 4H). At high multiplicities of infection, Shigella is cytotoxic to human, but not murine neutrophils (Francois et al., “Induction of Necrosis in Human Neutrophils by Shigella Flexneri Requires Type In Secretion, IpaB and IpaC Invasins, and Actin Polymerization,” Infect Immun 68:1289-1296 (2000), which is hereby incorporated by reference). In human neutrophils where neutrophil elastase was blocked with ICI-200355 and Shigella colonized the cytoplasm, neutrophil cytotoxicity was enhanced (FIG. 41). As expected, the noninvasive strain was not cytotoxic. Taken together, these data support a prominent role for neutrophil elastase in controlling Shigella infections.

The structural basis of the exquisite sensitivity of virulence proteins to neutrophil elastase is not known. Common structural patterns for virulence factors have been proposed (Pallen et al., “Coiled-Coil Domains in Proteins Secreted by Type III Secretion Systems,” Mol Microbiol 25:423-425 (1997); Miao et al., “Salmonella Typhimurium Leucine-Rich Repeat Proteins are Targeted to the SPI1 and SPI2 Type III Secretion Systems,” Mol Microbiol 34:850-864 (1999), which are hereby incorporated by reference) and might serve as recognition sites for neutrophil elastase action.

The ability of neutrophils to sequester and kill bacteria is a critical aspect of their defense function. This study constitutes the first demonstration of a previously unrecognized ability of neutrophil elastase to degrade type III secreted and surface exposed virulence factors of enterobacteria. The novel ability of neutrophil elastase to rapidly destroy these virulence factors suggests that this enzyme plays an important role in disarming pathogens.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

1. A method of treating bacterial infection in a subject comprising:
administering an elastase to the subject under conditions effective to target virulence factors from pathogenic bacteria.

2. The method according to claim 1, wherein the elastase is a neutrophil elastase.

3. The method according to claim 1, wherein the elastase is whole elastase or fragments thereof.

4. The method according to claim 3, wherein the elastase is whole elastase.

5. The method according to claim 3, wherein the elastase is a fragment of whole elastase selected from the group consisting of a His-Asp-Ser catalytic domain and a carbohydrate side chain.

6. The method according to claim 1, wherein the pathogenic bacteria are enterobacteria.

7. The method according to claim 6, wherein the enterobacteria is a Shigella species, a Salmonella species, or a Yersinia species.

8. The method according to claim 1, wherein the bacteria is a Chlamydia species, Pseudomonas aeruginosa, or a plant pathogenic bacteria.

9. A method of degrading bacterial virulence factors comprising:
subjecting the bacterial virulence factors to an elastase under conditions effective to degrade the bacterial virulence factors.

10. The method according to claim 9, wherein the elastase is a neutrophil elastase.

11. The method according to claim 9, wherein the elastase is whole elastase or fragments thereof.

12. The method according to claim 11, wherein the elastase is whole elastase.

13. The method according to claim 11, wherein the elastase is a fragment of whole elastase selected from the group consisting of a His-Asp-Ser catalytic domain and a carbohydrate side chain.

14. The method according to claim 9, wherein the bacteria is an enterobacteria.

15. The method according to claim 14, wherein the enterobacteria is a Shigella species, a Salmonella species, or a Yersinia species.

16. The method according to claim 9, wherein the bacteria is a Chlamydia species, Pseudomonas aeruginosa, or a plant pathogenic bacteria.

17. The method according to claim 9, wherein said subjecting is carried out at a concentration of at least 1.2 nM of the elastase.

18. The method according to claim 9, wherein said subjecting is carried out in vivo.

19. The method according to claim 9, wherein said subjecting is carried out in vitro.

20. A method of preventing bacteria from escaping phagosomes of neutrophils comprising:
subjecting the bacteria to an elastase under conditions effective to prevent the bacteria from escaping the phagosomes of the neutrophils.

21. The method according to claim 20, wherein the elastase is a neutrophil elastase.

22. The method according to claim 20, wherein the elastase is whole elastase or fragments thereof.

23. The method according to claim 20, wherein the elastase is whole elastase.

24. The method according to claim 20, wherein the elastase is a fragment of whole elastase selected from the group consisting of a His-Asp-Ser catalytic domain and a carbohydrate side chain.

25. The method according to claim 20, wherein the bacteria is a Shigella species, a Salmonella species, or a Yersinia species.

26. The method according to claim 20, wherein said subjecting is carried out at a concentration of at least 1.2 nM of the elastase.

27. The method according to claim 20, wherein said subjecting is carried out in vivo.

28. The method according to claim 20, wherein said subjecting is carried out in vitro.

29. The method according to claim 20, wherein said subjecting targets virulence factors from pathogenic bacteria and inactivates the bacteria.

30. A method of preventing bacteria from invading host cells comprising:
subjecting the bacteria to an elastase under conditions effective to prevent the bacteria from escaping invading host cells.

31. The method according to claim 30, wherein the elastase is a neutrophil elastase.

32. The method according to claim 30, wherein the elastase is whole elastase or fragments thereof.

33. The method according to claim 30, wherein the elastase is whole elastase.

34. The method according to claim 30, wherein the elastase is a fragment of whole elastase selected from the group consisting of a His-Asp-Ser catalytic domain and a carbohydrate side chain.

35. The method according to claim 30, wherein the bacteria is a Shigella species, a Salmonella species, or a Yersinia species.

36. The method according to claim 30, wherein said subjecting is carried out at a concentration of at least 1.2 nM of the elastase.

37. The method according to claim 30, wherein said subjecting is carried out in vivo.

38. The method according to claim 30, wherein said subjecting is carried out in vitro.

39. The method according to claim 30, wherein said subjecting targets virulence factors from pathogenic bacteria and inactivates the bacteria.
